(19) United States
(12) Patent Application Publication
(10) Pub. No.: US 2006/0124137 A1
(43) Pub. Date: Jun. 15, 2006

(54) Isometric exercise method and program for treating sleep apnea and snoring

(76) Inventor: Walter Fong, Fountain Valley, CA (US)

Correspondence Address: GENE SCOTT; PATENT LAW & VENTURE GROUP, 3140 RED HILL AVENUE, SUITE 150, COSTA MESA, CA, 92626-3440, US

(21) Appl. No.: 11/349,450

(22) Filed: Feb. 6, 2006

Related U.S. Application Data
(63) Continuation-in-part of application No. 10/855,692, filed on May 26, 2004.

(51) Int. Cl. A61F 5/56; A61C 5/14

(52) U.S. Cl. 128/848; 128/200.24; 128/859; 128/860

(57) ABSTRACT

Isometric muscle manipulation results in a reduction in breathing airway restriction and the incidence and magnitude of sleep apnea and snoring. The method comprises exercises including, raising the tip of the tongue upward and backward in the mouth to touch the area between the hard palate and the soft palate of the mouth and then pressing the tongue upwardly, raising the flat tongue upward pressing it against the hard palate, pressing the tongue against the floor of the mouth, protruding the jaw into a forward position, pressing the protruding lips together, and pressing the soft pallet against the rear surface of the tongue.

### CROSS-REFERENCE TO RELATED APPLICATIONS

This is a continuation-in-part application of U.S. patent application Ser. No. 10855,692, filed May 26, 2004, and which is incorporated herein by reference.

### STATEMENT REGARDING FEDERALLY SPONSORED RESEARCH OR DEVELOPMENT

Not applicable.

### THE NAMES OF THE PARTIES TO A JOINT RESEARCH AGREEMENT

Not applicable.

### INCORPORATION-BY-REFERENCE OF MATERIAL SUBMITTTED ON A COMPACT DISC

Not applicable.

### REFERENCE TO A “MICROFICHE APPENDIX”

Not applicable.

### BACKGROUND OF THE INVENTION

1. Field of the Present Disclosure

This disclosure relates generally to methods for treating sleeping disorders and more particularly to a method for treating obstructive sleep apnea and snoring through the use of exercises for strengthening muscles related to air flow health.

2. Description of Related Art Including Information Disclosed Under 37 CFR 1.97 and 1.98

Sleep apnea is a serious and potentially deadly disease affecting a significant portion of the population. However, relatively little research had been done on the cause and treatment of this illness. Apnea was first described in 1965 and characterized as a brief interruption of breathing patterns accompanied by snoring during sleep. There are varying degrees of apnea differentiated by the frequency of periodic breathing cessation episodes during sleep, and also the degree of hypoxia resulting from the related irregular breathing pattern.

When normal breathing is disrupted during sleep, an increased carbon dioxide level and a reduced oxygen level appears in the blood. The sleeping person is then suddenly aroused and gasps for air, and this is often accompanied by a loud snort. Sleep apnea has been variously described as choking, coughing, and sudden awakening with lack of breath, and usually with snoring.

An increased carbon dioxide level in the blood can cause adverse affects on the vital organs. The heart is usually caused to pump harder and at a higher rate in an effort to compensate for the lack of oxygen. Other organs, such as the kidneys and liver are suddenly confronted with increased blood flow and must adjust to cope with this change. This cycle may be repeated many times during a night's rest and often results in a general lack of proper rest and the restorative benefits thereof. Some of the health problems associated with sleep apnea include hypertension, heart attack, stroke, irregular heart beat as well as psychogenic conditions from the loss of restful sleep.

Within the medical community a common belief as to the cause of this condition is a narrowing of the airways while sleeping. This is believed to be caused by a relaxation of the tissue structures surrounding the airway. However, what tissue structures should the condition be attributed to is still not fully agreed upon. Also, there is no consensus as to why certain tissue structures close. Certainly, we know that the conformation of apnea related tissue structures vary between individuals and aging characteristics of these structures also vary greatly between individuals. Such variations may underlie the fact that apnea onset may occur even in youth as well as in the aged.

Medical treatment for sleep apnea has varied widely and much has been experimental. A person's health and age have been strong considerations as to what approach to take. Medication is often ineffective. Mechanical therapy providing for continuous positive airway pressure is the most common form of treatment. This procedure requires the patient to wear a facial mask during sleep while a constant pressure from an air blower forces air into the nasal passageway. The principle of this approach is that forced air flow tends to keep the throat from closing during sleep when muscles are relaxed. Dental appliance have been used to advantageously position the lower jaw and tongue, but have had a low success in improving milder cases of apnea.

Surgery has been used to widen the airway but has produced less than satisfactory results. For instance uvulopalatopharyngoplasty is a procedure to remove the tonsils, uvula, and a portion of the soft palate. Laser-assisted uvulopalotopharyngoplasty has been found to help in the reduction of snoring but is much less effective in reducing apnea. Somnoplasty is an investigative procedure somewhat effective in reducing the uvula and the back of the tongue using radiowaves. Tracheotomy is used on patients with severe and life-threatening apnea. In this procedure, a small opening in the trachea is made and a tube is inserted to provide a direct air vent to the lungs.

Other procedures involve psychological counseling and life style changes on patients who are overweight, and for those consuming significant amounts of alcohol and other control substances, as well as patients using sleeping pills. It is commonly believed that these substances contribute to sleep apnea. The following art defines the present state of technical approaches in the field of this invention.

Lubit, U.S. Pat. No. 3,447,534 describes a method of improving functioning soft palate tissue in preventing escape of air through the nose during speaking, which comprises positioning an expansible contractible element in the mouth adjacent to soft palate tissue, said element being adapted to press against the tissue of the soft palate and the posterior pharyngeal wall in its expanded condition and alternately expanding and contracting said element to stretch and exercise the muscles of the tissues of the soft palate and posterior pharyngeal wall.

Berry, U.S. Pat. No. 4,133,306 describes a semi-manually operated stimulus delivery mechanism incorporating various timing, measuring, counting and visual signal devices which control according to preset criteria, inflation and deflation of an air-filled oral exercise prosthesis.

Light, U.S. Pat. No. 5,213,553 describes a complemental series of handheld, tactile devices in kit form to assist in behavioral modification training techniques as well as utilizing the techiques of “resisted movement” and “successive approximation”. Each positioner device is handheld, with one portion protruding from the lips and the other portion loosely fitting on the patient's hard plate. Different shapes are introduced to the tongue and lips on each respective device. A commonalty within the interdependent positioner devices is presented by an elongated palatal base defining a convex upper surface to engage the palate, a manipulable handle projecting from the base and the respective devices are further characterized in configuration. Variations in configuration such as elongation, thickenings and apertures of the positioners involve primary modification to the palatal base and adjacent interconnection of the handle, whereby upon activation the patient may exercise his tongue and lips to achieve consonantal speech sounds, perfect swallowing techniques, and aid in mastication.

Miller, U.S. Pat. No. 5,431,610 describes a facial muscle exercise device including a mouthpiece which fits into the mouth over the lower jaw and teeth, which mouthpiece is connected via an elastic member to a handle which is pressed down by the user to create tension against the lower jaw. The lower jaw may then be exercised by up and down motion or thrusting forward and back or from side to side. Proper use of this device will cause the muscles in the face, chin and neck to become toned and conditioned, eliminating fatness below the chin and wrinkles.

Miller, U.S. Pat. No. 5,501,646 describes a jaw and neck muscle exercise apparatus, which includes a spring loaded support arm attached to a soft chin support on one end and to a chest plate on the other end. The chest plate is strapped around the chest bone/collar bone area and the back of the neck by a collar strap. The lower jaw may then be exercised by opening the mouth or lowering the entire head against the resistance of the spring. The resistance is selected to be proper for the size and strength of the user and the progress of the exercise program to date. Proper use of this device will cause the muscles of the face, chin and neck to become toned and conditioned, eliminating fatness below the chin and wrinkles, particularly exercising the hyoid, the infrahyoid and digastric muscles.

Karell, U.S. Pat. No. 5,792,067 describes an electromuscular stimulator that effects a beneficial medical purpose selected from the group consisting of mitigating snoring, mitigating obstructive sleep apnea, mitigating hypertension, dental analgesia, general analgesia, monitoring physiological conditions and facilitating the intraoral delivery of medication is disclosed. The electromuscular stimulator includes a first electrode for making electrical contact with a first anatomical structure selected from the group consisting of a hard palate, a soft palate and a pharynx; a second electrode for making electrical contact with a second anatomical structure; a control unit operably connected to the first and second electrodes; and a means for positioning the first and second electrodes relative to the first and second anatomical structures, respectively.

Shafer, U.S. Pat. No. 5,855,535 describes a compact jaw muscle exercise device that people use to strengthen and tone jaw muscles which includes a generally wedged shape body of plastic having upper and lower surfaces adapted to engage one's teeth. Holes run transversely through the body, which increases the deformability of the exercise device. The exercise device's resistance may be increased by inserting cylindrical members through the holes.

Horstel et al., U.S. Pat. No. 6,471,621 describes a method of treatment of groups of muscles in orofacial region, of the respiratory system, and of retaining and support system of a body of a patient and including providing a logopedic aid having at least one rubbed balloon and a plurality of valve mouthpieces for inflating the at least one rubbed balloon and characterized by different degrees of difficulty with which the balloon can be inflated, and treating the patient by having the patient inflate the balloon by using in succession valve mouthpieces with an ever increasing degree of difficulty.

Leelamanit et al., U.S. Pat. No. 6,484,053 describes a sequential stimulator for the treatment of dysphagic patients that incorporates a unit, which is capable of detecting a swallowing signal, from the glossal or temporalis surface electromyography (SEMG). When a swallowing signal is recognized, a trigger signal is sent to the stimulation generation unit to release high voltage stimuli sequentially to the suprahyoid muscles or the masseter muscles and the pharyngeal muscles in order to assist in the elevation of the larynx. This enables the pharyngeal lumen to open more widely so that food can pass through the patient's pharynx and into the oesophagus more easily during swallowing. Thus the sequential stimulator is a device for assisting swallowing in patients with dysphagia due to a variety of causes, for instance, brain injury, cerebrovascular accident, injury of the cervical nerves, muscles weakness, or old age. The stimulator is operative only when the patient attempts to swallow and provides a physiologic stimulus and provides a means of immediate relief of the swallowing difficulty. The device is also useful for physical therapy whereby the muscles under the chin, the masseter muscles and the pharyngeal muscles can be re-educated to contract in the normal coordinated sequence.

Arias, U.S. Pat. No. 6,524,225 describes a jaw, jowl and neck exercise kit to exercise the following muscles: masseters, temporalis, pterygoids, digastric, depressor anguli oris, depressor labii inferioris, mentalis, platysma and suprahoid muscles: mylohyioid, hyoglossus, geniiohyiod, genioglossus, Buccinator, Zygomaticus mayor, Zygomaticus minor, Risorius and Orbicularis oris non-invasively and without the use of electronics. The jaw, jowl and neck exercise kit comprises instruments that allow the user to isolate and contract the face and neck muscles. These contractions cause the aforementioned muscles to be exercised in a manner to produce efficient and fast results. The jaw, jowl and neck exercise kit is comprised of instruments designed to target different muscles and to allow the user to gradually increase resistance and to improve the effect of the exercise.

Ellis, U.S. Pat. No. 6,561,188 describes an apparatus configured to provide improved nasal breathing to persons requiring a range of respiratory based remedies ranging from opening collapsed nasal passages to treating allergic reaction to delivery of immediate dosage or controlled release medication. The apparatus includes connected nasal inserts which provide nodules to open the nasal passage and which directly align with the nasal air channel. For a preferred embodiment which provides a means to delivery of breathable gas mixtures or nasal and sinus rinse solutions, the inserts contain cannula which receive and connect to the source of the gas or rinse. For another embodiment using filtration, the inserts receive disposable filtration pacs. Sustained release of predetermined medical dosages is obtained by a further embodiment wherein the inserts receive medication inserts.

Byers, U.S. Pat. No. 6,652,275 describes an intra-oral article that is user-customized from a contoured, thermoplastic pre-form stretches and reconfigures cheek skin, thereby diminishing the appearance of cheek lines, wrinkles, folds, and depressions. After heating a pre-form to its softening point, it is pressed against the buccal surface of the gums and teeth in a posterior quadrant of the maxilla or mandible. The pre-form is removed from the mouth after hardening, which yields a customized impression on the interior surface of the pre-form. The customized article can subsequently be applied and removed as desired by the user.

Joubert et al., U.S. H1557 describes an arrangement for rewarding tongue-retraction exercise therapy performed by swallowing-impaired surgical and disease patients is disclosed. The achieved therapy includes execution of an oral sucking act (as opposed to lung originated suction) in combination with provision of a patient communicated incentive feedback signal measurement of the sucking act pressures-pressures below atmospheric pressure. The patient communication includes a visual bar graph related display and in the disclosed apparatus is provided with a plurality of four scale ranges in order to accommodate patients of varying swallowing act impairment. The achieved base of tongue strengthening exercise has been found to enable swallowing recovery in weeks in lieu of the expected months (or never) that is normally seen in severely impaired surgical or disease patients. A discussion of the swallowing act and its impairment behavior is included.

Our prior art search with abstracts described above teaches various apparatus with methods of using the apparatus for exercising to improve muscle coordination and strength for a variety of dysfunctional issues. This literature includes: a method and device for massaging the soft palate, a palatal exerciser control device, devices used to improve speech, swallowing and mastication, a facial muscle exerciser apparatus, a jaw, face and neck muscle exercise apparatus, an apparatus and method for mitigating sleep and other disorders through electromuscular stimulation, a jaw muscle exercise device, a method of treatment of groups of muscles in an oral-facial region by using an inflatable rubber balloon as a logopedic aid, a method and apparatus for treating poor laryngeal elevation disorders with sequential high voltage electrical stimulation, a maxillary occlusion muscles exerciser, cheek muscles exerciser, and a cheek and mouth muscles exerciser, a nasal breathing apparatus and methods of use, an intra-oral article for cosmetically stretching and reconfiguring cheek skin and method for customizing same, and a method for swallowing rehabilitation. However, the prior art does not teach a muscle training and conditioning technique for treating sleep apnea and snoring without the need of associated hardware, devices, or apparatus. The present invention fulfills this need and provides further related advantages as described in the following summary.

### BRIEF SUMMARY OF THE INVENTION

This disclosure teaches certain benefits in construction and use which give rise to the objectives described below.

In a best mode embodiment of the present invention, isometric muscle manipulation results in a reduction in breathing airway restriction and in the incidence and magnitude of sleep apnea and snoring. The method comprises exercises including, pressing the tongue against various parts of the mouth in a series of presses with the teeth held clenched and further includes pressing the lips together, and moving the lower jaw to an extended forward position as will be described in further detail herein. The present program was developed by Dr. Walter Fong, D.C., F.I.A.C.A., F.C.T.S., who is a registered radiology supervisor, a certified acupuncturist for laser and electronic acupuncture, a certified thermographer and an accomplished inventor. The particular set of isometric exercises carried out in the program described herein has been found to accomplish the following objectives. In this regard, patients are asked to provide a brief history of their snoring and sleep apnea habits including frequency, intensity and when the condition was initiated to determine its duration. A subjective grade between 1 and 10 is assigned to each patient to indicate the level of snoring, i.e., a snoring index. Patients are then trained in the presently disclosed drills and program. Patients keep a daily diary, see FIG. 4 in the accompanying figures. Usually, between about 2 to 4 weeks of performing the program on a daily bases, the muscles that are related tend to show improvements in tone and strength and this is indicated by the ease of which the exercises are conducted. Time and repetition of the exercises are increases accordingly on an incremental bases. When snoring and sleep apnea has reached a satisfactory reduction, the patient is placed on a maintenance program.

A primary objective of one embodiment of the present invention is to provide a method of exercising that yields advantages not taught by the prior art.

Another objective is to provide a method capable of reducing the incidence and degree of nocturnal snoring.

A further objective is to provide a method capable of reducing the incidence and degree of sleep apnea.

A further objective is to provide a method for reducing the incidence and degree of snoring and sleep apnea without the aid of an apparatus.

A still further objective is to provide a method of reducing the incidence and degree of snoring and sleep apnea without the need for taking conscious action just prior to, or during the sleeping period.

A yet further objective is to provide a method for reversing muscle degeneration and for strengthening genetically inferior, muscle structures and tissue conformations which are contributory to snoring and, or sleep apnea.

Other features and advantages of the embodiments of the present invention will become apparent from the following more detailed description, taken in conjunction with the accompanying drawings, which illustrate, by way of example, the principles of at least one of the possible embodiments of the invention.

Other features and advantages of the present invention will become apparent from the following more detailed description, taken in conjunction with the accompanying drawings, which illustrate, by way of example, the principles of the presently described apparatus and method of its use.

### BRIEF DESCRIPTION OF THE SEVERAL VIEWS OF THE DRAWING(S)

Illustrated in the accompanying drawing(s) is at least one of the best mode embodiments of the present invention In such drawing(s):

FIGS. 1-6 are vertical cross-sectional views of a human head showing the tongue, lips pallet and other features thereof whereby:

FIG. 1 shows an exercise wherein the tongue is pressed against the hard pallet;

FIG. 2 shows an exercise wherein the tongue is pressed against the roof of the mouth at a point between the hard pallet and the soft pallet;

FIG. 3 shows an exercise wherein the tongue is pressed against the lower mouth surface;

FIG. 4 shows an exercise wherein the lower jaw is pressed forward, i.e., jutted-out;

FIG. 5 shows an exercise wherein the lips are pressed together and jutted-out;

FIG. 6 shows an exercise wherein the back of the tongue is pressed against a lowered soft pallet; and

FIG. 7 is a data sheet for recording and analyzing progress in a program referred to as the “Silent Night” treatment.

### DETAILED DESCRIPTION OF THE INVENTION

The above described drawing figures illustrate the described apparatus and its method of use in at least one of its preferred, best mode embodiment, which is further defined in detail in the following description. Those having ordinary skill in the art may be able to make alterations and modifications what is described herein without departing from its spirit and scope. Therefore, it must be understood that what is illustrated is set forth only for the purposes of example and that it should not be taken as a limitation in the scope of the present apparatus and method of use.

The above described drawing figures illustrate the individual exercises of the present invention program representing the best mode embodiment of the present invention method, which is further defined in detail in the following description. Those having ordinary skill in the art may be able to make alterations and modifications in the present invention without departing from its spirit and scope. Therefore, it must be understood that the stated embodiments have been set forth only for the purposes of example and that they should not be taken as limiting the invention as defined in the following.

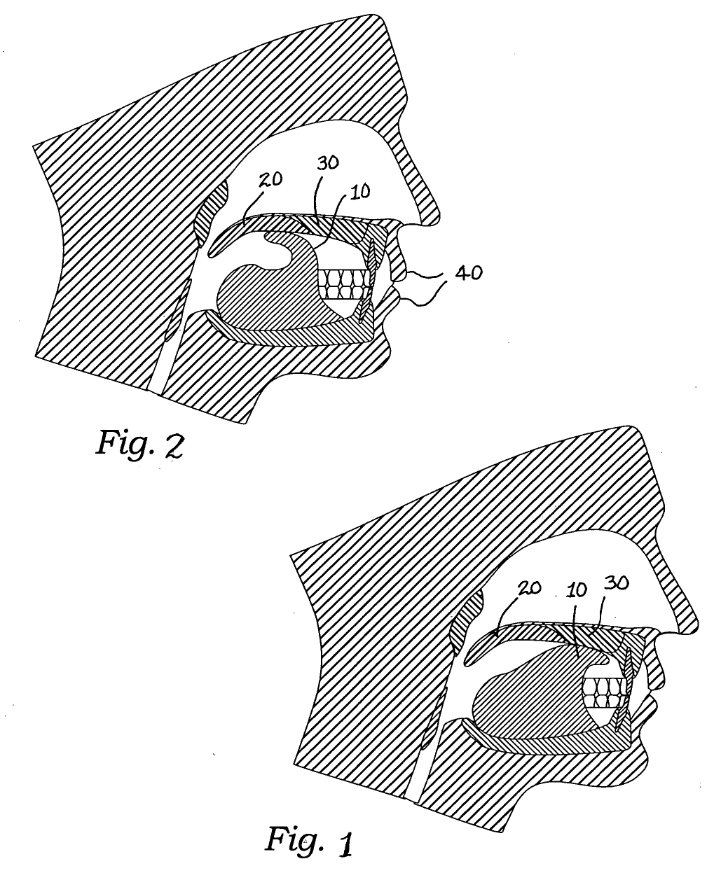

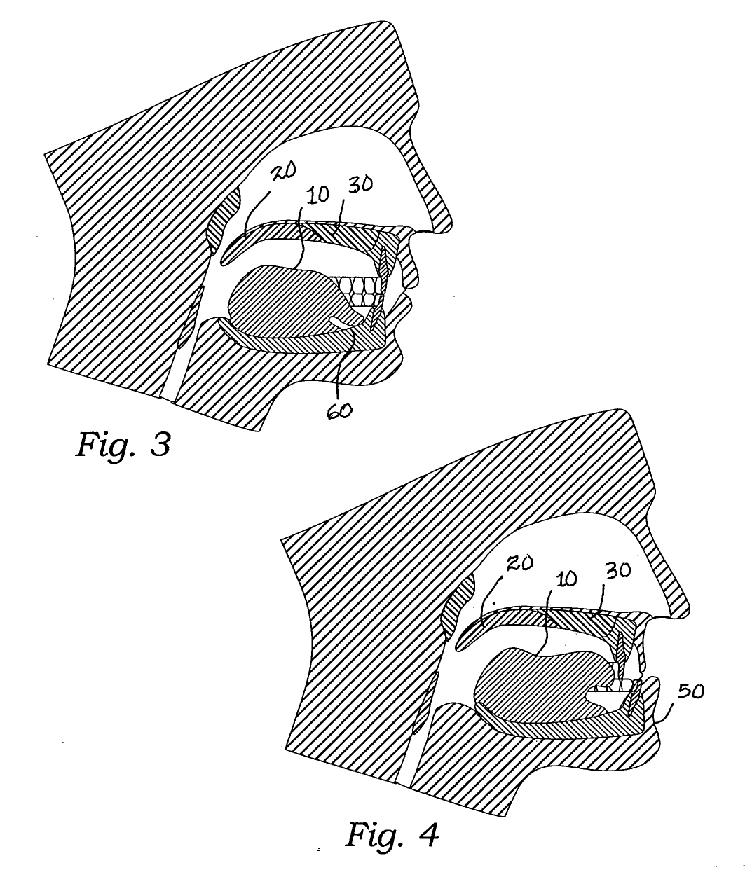

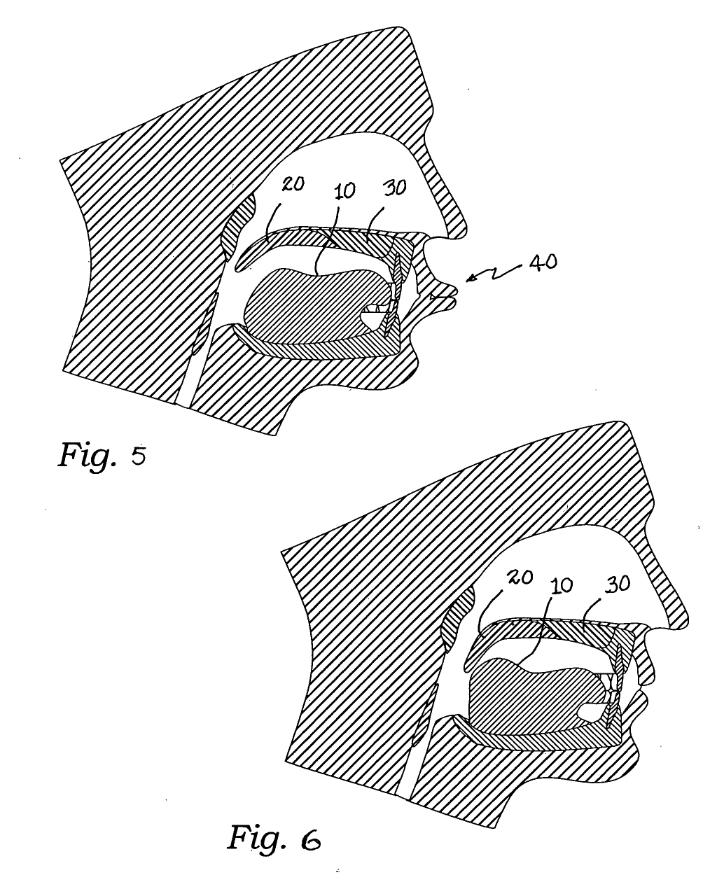

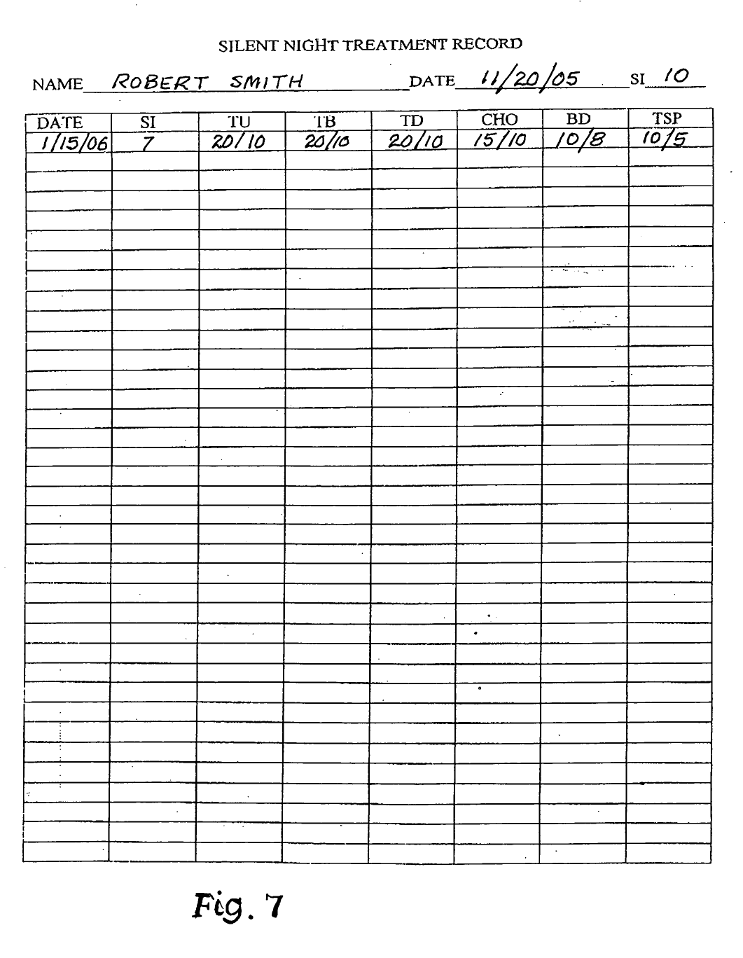

A program for treating snoring and sleep apnea is disclosed herein. As stated above, patients with chronic snoring and apnea problems are treated and results have been found to be very favorable. FIG. 7 is a data record sheet wherein the following abbreviations are defined as: SI=snore index, 1 to 10, 1 being best, TU=tongue up forward (FIG. 1), TB=tongue back (FIG. 2), TD=tongue down (FIG. 3), CHO=chin out (FIG. 4), BD=bite down with lips extended fully (FIG. 5, and TSP=tongue against soft pallet (FIG. 6). In SI is placed the snore index as estimated by the client's sleeping partner who is active in the present program. Each of the further related boxes, moving across the table, are filled in using the following format: X/Y, where X is the time in seconds that each repetition of the subject exercise is held and Y is the number of repetitions.

As examples of actual results obtained, several patients are herein characterized from actual patient records. These patients performed the instant isometric exercises which program is referred to in doctor's office as the “Silent Night Treatment,” under doctors supervision for at least two times daily and particularly at bed time. Patient 1 had a history of chronic constant loud snoring for years with occasional sleep apnea. After four weeks using the present method a complete cessation of snoring and apnea resulted. Patient 2 had a history of chronic loud snoring and the program resulted in a reduction to occasional light snoring. Patient 3 had chronic constant loud snoring and the program resulted occasional intermittent snoring. Patent 4 had a history of loud snoring with apnea and this was reduces to a complete cessation of apnea with only occasional snoring. Patient 5 had a history of chronic constant loud snoring with frequent apnea, and this was reduced to a full cessation of apnea but with intermittent light snoring. Patient 6 had a history of chronic constant loud snoring which was reduced to light snoring and heavy breathing. The six cases all showed significant improvement with none showing any side effects or difficulties due to the procedures of the present method.

In FIGS. 1-6, the following elements of the mouth and surrounding structure are identified by: tongue 10, soft pallet 20, hard pallet 30, lips 40, lower jaw 50, and lower mouth interior surface 60.

One aspect of a best mode embodiment of the present invention isometric muscle manipulation results in a reduction in breathing airway restriction and has been shown by trials to lead to a reduction in the incidence and magnitude of sleep apnea and snoring. The method comprises six critical exercises for building and strengthening important muscles involved in the maintenance of the human air passage. These exercises each may be practiced alone with some therapeutic benefit, but when practiced together as an exercise group in a program of increased difficulty, significant benefit is possible. Such benefit will vary from individual to individual depending on muscle strength and conformation, age, sleeping position and other variables, however, it has been found that the exercised defined here are important to the maintenance of good health for most individuals. In detail, the exercises are conducted with teeth touching and with stressing the muscles involved for between 3 and 10 seconds, without overstrain as follows: raising the tongue 10 upward against the hard palate 30 at the front of the mouth and then pressing the tongue against the hard palate (FIG. 1), raising the tongue 10 upward and backward in a mouth to press against the soft palate 20 along the roof of the mouth (FIG. 2), pressing the tongue 10 against a floor 60 of the mouth (FIG. 3), protruding the lower jaw 50 forwardly to its forward-most position (FIG. 4), pursing and pressing the lips 40 forward and together (FIG. 5), and lowering the soft pallet 20 to contact and press against the back of the tongue 10 (FIG. 6). Preferably, these exercises are repeated, in sets of ten repetitions, each with appropriate rests between sets. One may do all six exercises in sequence, as a single set, repeating this set a total of ten times, or one may do ten repetitions of each single exercise with short rests between each repetition and then move on to the next exercise, staging the sequence of exercises at random or by selection. These two approaches are shown below, where numerals represent each of the six exercises.

| | |
|---|---|
| First set | 1, 2, 3, 4, 5, 6 |
| Second set | 1, 2, 3, 4, 5, 6 |
| Third set | 1, 2, 3, 4, 5, 6 |
| Fourth set | 1, 2, 3, 4, 5, 6 |
| Fifth set | 1, 2, 3, 4, 5, 6 |
| Sixth set | 1, 2, 3, 4, 5, 6 |
| Seventh set | 1, 2, 3, 4, 5, 6 |
| Eighth set | 1, 2, 3, 4, 5, 6 |
| Ninth set | 1, 2, 3, 4, 5, 6 |
| Tenth set | 1, 2, 3, 4, 5, 6 |
| First set | 1, 1, 1, 1, 1, 1, 1, 1, 1, 1 |
| Second set | 2, 2, 2, 2, 2, 2, 2, 2, 2, 2 |
| Third set | 3, 3, 3, 3, 3, 3, 3, 3, 3, 3 |
| Fourth set | 4, 4, 4, 4, 4, 4, 4, 4, 4, 4 |
| Fifth set | 5, 5, 5, 5, 5, 5, 5, 5, 5, 5 |
| Sixth set | 6, 6, 6, 6, 6, 6, 6, 6, 6, 6 |

With respect to the exercises described above, certain critical muscles groups are strengthened through flexing isometrically, with the antagonistic muscles providing resistance in an isometric manner. The muscles used include the stylopharyngeus muscles, tensor veli palatine muscles, the palatopharyngeus muscles, the tongue itself and the salpingopharyngeus muscles, the musculus uvulae and the oral pharynx 80 to mention just a few of the muscle groups effected. The exercising of these four muscle groups results in strengthening of these muscles and their surrounding tissues which results in improving and maintaining clearances in the human mouth and throat so as to widen and maintain a clear air passage even when sleep tends to relax all muscle tissues. The holding of isometric force against the resistance of the antagonistic muscles for between 3 and 10 seconds duration has been found to be critical to the success of the exercises and results in a balance between exercise benefit versus exercise fatigue. A person using these exercises may find that at first a 3 second isometric duration results in fatigue, but through repetition and daily workout, the duration can be made to extend for up to 10 seconds each for 10 repetitions with a few seconds of rest between repetitions. Of all possible combinations of muscle exercises of the mouth and throat muscle groups, it has been found that the present selection is superior to any other choice in bringing fast results and above all, continued improvement. Selections of less then the six described exercises have been tested with much inferior results or no improvement in chronic apnea problems. However, it has been found that the selected six exercises, when used as a unitary combination, results in great improvement with a lessening or cessation in snoring and with significant improvement in the number of apnea episodes and the duration of such episodes. Patients with heavy vibratory snoring have been brought to a level where such snoring noise is light with only the sound of the flow of air through the throat, but without pharyngeal contribution. Patients with ten to twenty breathing cessation episodes per hour of sleep have been brought to only one or two such episodes; and breathing cessation durations of from 20 to 40 seconds, have been reduces to 5 seconds or less. It has been found that a synergistic effect is achieved when all five of the stated exercises are performed together and it is thus this synergy and its benefit that compels the user to apply the present inventive method as one step in health improvement.

The enablements described in detail above are considered novel over the prior art of record and are considered critical to the operation of at least one aspect of the apparatus and its method of use and to the achievement of the above described objectives. The words used in this specification to describe the instant embodiments are to be understood not only in the sense of their commonly defined meanings, but to include by special definition in this specification: structure, material or acts beyond the scope of the commonly defined meanings. Thus if an element can be understood in the context of this specification as including more than one meaning, then its use must be understood as being generic to all possible meanings supported by the specification and by the word or words describing the element.

The definitions of the words or drawing elements described herein are meant to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements described and its various embodiments or that a single element may be substituted for two or more elements in a claim.

Changes from the claimed subject matter as viewed by a person with ordinary skill in the art, now known or later devised, are expressly contemplated as being equivalents within the scope intended and its various embodiments. Therefore, obvious substitutions now or later known to one with ordinary skill in the art are defined to be within the scope of the defined elements. This disclosure is thus meant to be understood to include what is specifically illustrated and described above, what is conceptually equivalent, what can be obviously substituted, and also what incorporates the essential ideas.

The scope of this description is to be interpreted only in conjunction with the appended claims and it is made clear, here, that each named inventor believes that the claimed subject matter is what is intended to be patented.

What is claimed is:

1. An isometric exercise program leading to the cessation of snoring comprising the steps of:
a) pressing the tongue against the hard palate with the teeth touching;
b) pressing the tongue against the soft palate with teeth touching;
c) pressing the tongue against the floor of the mouth with teeth touching;
d) protruding the jaw forwardly with teeth touching while tightening the muscles of the jaw, chin and neck simultaneously;
e) puckering and pressing the lips together with teeth touching; and
f) pressing the soft palette against the tongue with teeth touching;
wherein each of the steps is conducted for at least 3 seconds.

2. The exercise program of claim 1 wherein steps (a) through (f) are repeated in sets.

3. The method of claim 2 wherein each of the exercises (a) through (f) is performed repeatedly for up to 10 repetitions followed by each of the next exercises (a) through (f) until all of the exercises (a) through (f) have been completed.

4. The method of claim 2 wherein each the exercises (a) through (f) is performed in sequence to complete a set, followed by at least 10 repetitions of the set.

5. The isometric exercise program of claim 1 conducted until snoring has reached an acceptable level and then followed by conducting a maintenance program using step (d) alone.

6. An isometric exercise program leading to the cessation of snoring comprising at least one of the steps:
a) pressing the tongue against the hard palate with the teeth touching;
b) pressing the tongue against the soft palate with teeth touching;
c) pressing the tongue against the floor of the mouth with teeth touching;
d) protruding the jaw forwardly with teeth touching while tightening the muscles of the jaw, chin and neck simultaneously;
e) puckering and pressing the lips together with teeth touching; and
f) pressing the soft palette against the tongue with teeth touching;
wherein the at least one of the steps is held for plural intervals of at least 3 seconds each with the intervals interspersed with further intervals of relaxation therebetween.

7. The isometric exercise program of claim 6 conducted until snoring has reached an acceptable level and then followed by conducting a maintenance program using step (d) alone.
